(19) United States
(12) Patent Application Publication
(10) Pub. No.: US 2019/0022350 A1
(43) Pub. Date: Jan. 24, 2019

(54) SYSTEM FOR INDUCING SLEEP OF INFANT

(71) Applicant: Sae Han Tungsten Co., Ltd., Namyangju-si, Gyeonggi-do (KR)

(72) Inventors: Jung Hyun SEO, Uijeongbu-si, Gyeonggi-do (KR); Eunyoung CHO, Namyangju-si, Gyeonggi-do (KR); Kangbok LEE, Namyangju-si, Gyeonggi-do (KR); Hak Jun KIM, Seoul (KR)

(73) Assignee: Sae Han Tungsten Co., Ltd., Namyangju-si, Gyeonggi-do (KR)

(21) Appl. No.: 15/752,591

(22) PCT Filed: May 30, 2017
(86) PCT No.: PCT/KR2017/005613

§ 371 Date: Feb. 14, 2018

(30) Foreign Application Priority Data
Apr. 14, 2017 (KR) 10-2017-0048709

(51) Int. Cl. A61M 21/02

(52) U.S. Cl. CPC A61M 21/02; A61M 2021/0027; A61M 2230/04

(57) ABSTRACT

A system for inducing sleep of an infant includes: a cushion main body, an operation main body unit provided inside the cushion main body, a measurement means connected to the operation main body unit to measure heartbeat of a human body, and a switch unit, wherein the measurement means includes a hollow case opened toward one side and a measurement unit provided in an opening of the case to measure the heartbeat; the operation main body unit includes a main body unit, a control unit provided in the main body unit, a storage unit connected to the control unit and provided in the main body unit, an output unit connected to the control unit and provided in the main body unit, and a display unit connected to the control unit and provided in the main body unit; and the switch unit is provided to be connected to the control unit.

### TECHNICAL FIELD

The present invention relates to a system for inducing sleep of an infant, and more specifically, to a system for inducing sleep of an infant, which effectively induces sleep of a baby and extends sleep state duration.

### BACKGROUND ART

Generally, an infant who has developed vitality for ten months in the mother's womb while listening regular heartbeat sounds will lose sense of psychological stability if he or she is separated from the mother. A baby having such a sensitive character like this may stably take a sleep only when he or she stays together with the mother.

However, it is difficult for the mother to hold the baby in the arms and induce a sleep every time she puts the baby to sleep, and although the baby falls asleep, the sleeping state is not continued if the baby is separated from the mother.

### DISCLOSURE OF INVENTION

### Technical Problem

Therefore, the present invention has been made in view of the above problems, and it is an object of the present invention to provide a system for inducing sleep of an infant, which effectively induce sleep and extend sleep state duration by giving the baby, by means of heartbeat sounds of the mother, a sense of psychological stability that the mother is staying together with the baby.

### Technical Solution

To accomplish the above object, according to one aspect of the present invention, there is provided a system for inducing sleep of an infant, the system comprising: a cushion main body, an operation main body unit provided inside the cushion main body, a measurement means connected to the operation main body unit to measure heartbeat of a human body, and a switch unit, in which the measurement means includes a hollow case opened toward one side and a measurement unit provided in the opening of the case to measure the heartbeat; the operation main body unit includes a main body unit, a control unit provided in the main body unit, a storage unit connected to the control unit and provided in the main body unit, an output unit connected to the control unit and provided in the main body unit, and a display unit connected to the control unit and provided in the main body unit; the switch unit is provided to be connected to the control unit; and a heartbeat signal of the human body measured by the measurement means is stored in the storage unit, and outputted and repeatedly played back through the output unit by a playback command of a user received through the switch unit.

In the system as described above, the switch unit includes a power switch, a mode change switch and a volume control switch, in which if a measurement mode is selected by operating the mode change switch, whether the heartbeat is stabilized is determined by measuring the heartbeat signal measured by the measurement means and operating the measured heartbeat signal, and a result of the determination is displayed through the display unit.

In the system as described above, if the user switches the mode change switch to a playback mode, the stabilized heartbeat signal measured and stored in the storage unit is repeatedly played back through the output unit.

In the system as described above, the heartbeat signal is stored in the storage unit by way of a stabilization determination step, and at the stabilization determination step, the heartbeat signal measured by the measurement means is operated, and a result of the operation is compared with a set value stored in the storage unit, and if the result of the operation is within a range of the set value stored in the storage unit, a storing indication signal is displayed by the display unit, and the heartbeat signal is stored in the storage unit as a heartbeat signal to be played back, and if the result of the operation is out of the range of the set value, a re-measuring indication signal is displayed by the display unit, and a new heartbeat signal is measured again and operated.

In the system as described above, at the stabilization determination step, the heartbeat signal is measured during two set periods and stored in the storage unit, an average value and a standard deviation of the two heartbeat signals are calculated, and whether the heartbeat is stabilized is determined by comparing the average values and the standard deviations of the two heartbeat signals.

### Advantageous Effects

The system for inducing sleep of an infant according to the present invention has an effect of effectively inducing sleep of a baby and extending sleep state duration by giving the baby a sense of psychological stability that the mother is staying together with the baby by means of repeatedly providing the baby with stabilized heartbeat sounds of the mother.

### BRIEF DESCRIPTION OF THE DRAWINGS

FIG. 1 is a view schematically showing the configuration of a system for inducing sleep of an infant of the present invention.

FIG. 2 is a view schematically showing the configuration of an operation main body unit of the present invention.

FIG. 3 is a view schematically showing the configuration of a switch unit of the present invention.

FIG. 4 is a flowchart schematically illustrating a measurement mode process of a system for inducing sleep of an infant of the present invention.

FIG. 5 is a graph exemplarily showing heartbeat sounds measured by a measurement means according to an embodiment of the present invention.

### BEST MODE FOR CARRYING OUT THE INVENTION

Hereafter, the technical configuration and operation of a system for inducing sleep of an infant according to the present invention will be described in detail with reference to the accompanying drawings.

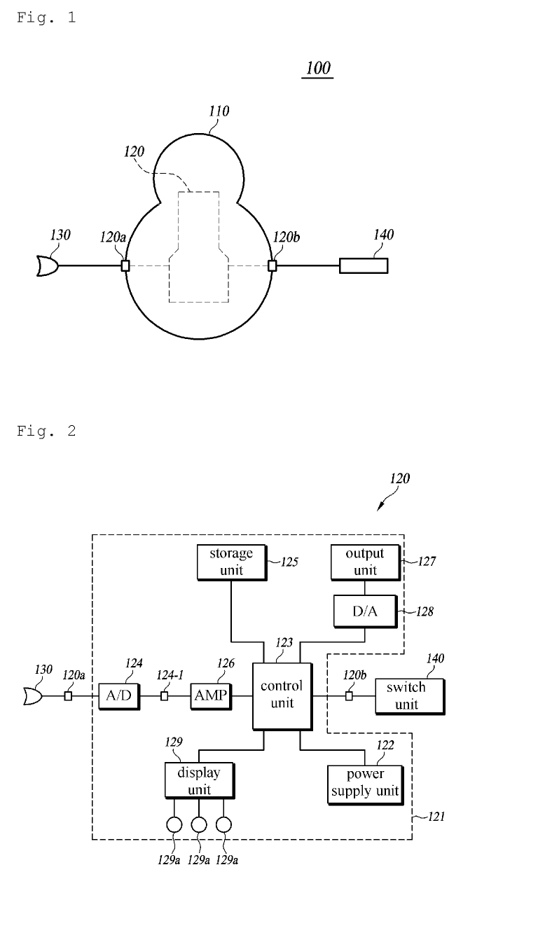

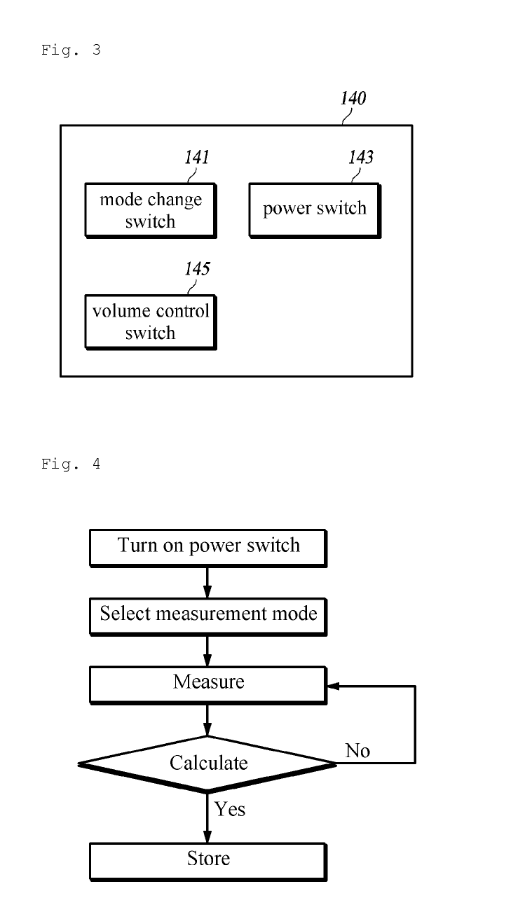

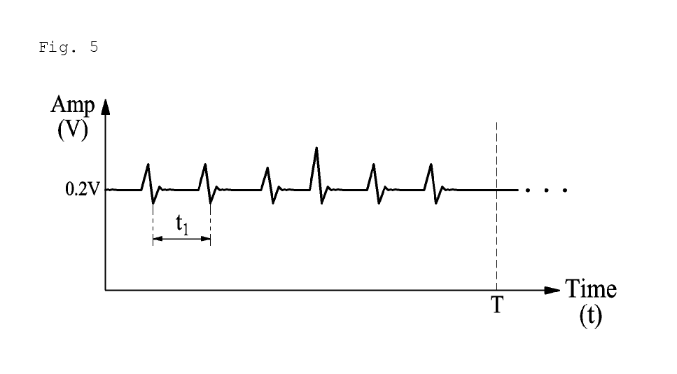

FIG. 1 is a view schematically showing the configuration of a system for inducing sleep of an infant of the present invention, FIG. 2 is a view schematically showing the configuration of an operation main body unit of the present invention, FIG. 3 is a view schematically showing the configuration of a switch unit of the present invention, FIG. 4 is a flowchart schematically illustrating a measurement mode process of a system for inducing sleep of an infant of the present invention, and FIG. 5 is a graph exemplarily showing heartbeat sounds measured by a measurement means according to an embodiment of the present invention.

The present invention proposes a system for effectively inducing sleep and extending sleep state duration by giving a baby a sense of psychological stability that the mother is staying together with the baby by means of repeatedly providing the baby with heartbeat sounds of the mother.

As shown in FIG. 1, a system for inducing sleep of an infant 100 according to the present invention includes a cushion main body 110, an operation main body unit 120 provided inside the cushion main body 110, a measurement means 130 connected to the operation main body unit 120 to measure heartbeat of a human body, and a switch unit 140 connected to the operation main body unit 120.

The cushion main body 110 is a product that can be provided near a baby when the baby sleeps, such as a cushion, a doll, a pillow or the like. Although a doll is explained as an example of the cushion main body 110 in the embodiment of the present invention, it is not limited thereto.

An open groove (not shown) is formed at one side of the cushion main body 110. The operation main body unit 120 is inserted into the groove and coupled to the cushion main body 110. A fabric cover which can be opened and closed to cover the open portion of the groove is provided in the cushion main body 110.

The operation main body unit 120 is provided inside the cushion main body 110. The operation main body unit 120 includes a main body unit 121, a power supply unit 122, a control unit 123, an A/D converter 124, an amplifier 126, a storage unit 125, an output unit 127, a D/A converter 128 and a display unit 129.

The main body unit 121 is formed as a hollow body having a space formed inside thereof. The power supply unit 122, the control unit 123, the A/D converter 124, the amplifier 126, the storage unit 125, the output unit 127, the D/A converter 128 and the display unit 129 are provided in main body unit 121. Main body connection terminals 120a and 120b for connecting the measurement means 130 and the switch unit 140 are installed in main body unit 121.

The power supply unit 122 includes a battery. The power supply unit 122 is connected to the control unit 123 and supplies power needed for the system for inducing sleep of an infant.

The A/D converter 124 is connected between the main body connection terminal 120a connected to the measurement means 130 and the control unit 123. Heartbeat sounds measured by the measurement means 130 are transferred to the control unit 123 by way of the A/D converter 124. The A/D converter 124 converts analog signals of the heartbeat sounds measured by the measurement means 130 into digital signals. A filter unit 124-1 may be further connected to the A/D converter 124.

The filter unit 124-1 performs an operation of passing only the sounds in a vocal range of 20 to 200 Hz, which is the range of general heartbeat sounds. Sounds other than the heartbeat sounds are filtered by the filter unit 124-1.

The amplifier 126 is connected between the A/D converter 124 and the control unit 123. The amplifier 126 amplifies the digitalized heartbeat signals transferred through the A/D converter 124 to facilitate operation thereof and transfers the amplified signals to the control unit 123.

The control unit 123 is connected to the power supply unit 122, the amplifier 126, the storage unit 125, the D/A converter 128, the display unit 129 and the main body connection terminal 120b connected to the switch unit 140 and controls operation thereof according to the process of progression or commands of the switch unit 140.

The storage unit 125 is connected to the control unit 123. The storage unit 125 stores heartbeat signals operated by the control unit 123 and determined as stabilized.

The output unit 127 is connected to the control unit 123 and outputs the sounds stored in the storage unit 125. The output unit 127 outputs heartbeat sounds corrected and amplified as a sound of a predetermined amplitude. For example, when a sound is outputted from the output unit 127, the sound is outputted after being amplified eight to twelve times as much as the measured sound. If the outputted heartbeat sounds are amplified to be less than eight times of the measured heartbeat sounds, it is difficult for the baby to recognize the heartbeat sounds, and if the outputted heartbeat sounds are amplified to exceed twelve times, the baby does not feel a sense of psychological stability. The output unit 127 is a speaker, and since it is a well-known technique, details thereof will be omitted.

The D/A converter 128 is connected between the output unit 127 and the control unit 123. The D/A converter 128 converts the heartbeat signals stored in the storage unit 125 so as to be outputted through the output unit 127. The D/A converter 128 converts digitalized heartbeat signals into analog signals.

The display unit 129 is connected to the control unit 123. The display unit 129 includes a plurality of signal lamps 129a, and an operation, a setting state or the like of the operation main body unit 120 is displayed to the outside. The display unit 129 may include a power lamp, a stabilization indication lamp, a playback lamp or the like.

The measurement means 130 is provided to be connected to the operation main body unit 120. The measurement means 130 includes a hollow case opened toward one side and a measurement unit provided in the opening of the case to measure the heartbeat.

The measurement unit contacts with the body of a user and measures heartbeat sounds. The measurement unit includes a microphone (a highly sensitive microphone sensor) and measures heartbeat in the principle of stethoscope. Since the technique of the microphone is a well-known technique, details thereof will be omitted.

A measurement connection terminal connected to the measurement unit and extended from the case is provided in the measurement means 130. The measurement connection terminal is connected to the main body connection terminal 120a of the operation main body unit 120. Accordingly, the measurement means 130 is detachable from the operation main body unit 120 and may be connected and used only when heartbeat is measured.

As shown in FIGS. 2 and 3, the switch unit 140 includes a power switch 143, a mode change switch 141 and a volume control switch 145. The user may control operation of the control unit 123 through the switch unit 140.

The switch unit 140 has a switch connection terminal and is connected to the main body connection terminal 120b of the operation main body unit 120. Accordingly, the switch unit 140 is detachable from the operation main body unit 120 and may be connected and used only when the control unit is controlled. The switch unit 140 may be provided to be integrated with the operation main body unit 120.

Describing the operation of the system for inducing sleep of an infant with reference to the contents as described above, the operation is divided into a measurement mode of storing heartbeat sounds of a mother and a playback mode of outputting the stored mother's heartbeat sounds.

FIG. 4 is a flowchart schematically illustrating a measurement mode process of a system for inducing sleep of an infant of the present invention, and FIG. 5 is a graph exemplarily showing heartbeat sounds measured by a measurement means according to an embodiment of the present invention.

As shown in FIGS. 4 and 5, power of the operation main body unit 120 is turned on through the power switch 143 of the switch unit 140.

The system for inducing sleep of an infant is switched to the measurement mode through the mode change switch 141 of the switch unit 140.

Heartbeat sounds are measured through the measurement means 130 contacting with the body of the mother, i.e., the user. As shown in the graph of FIG. 5, the measurement means 130 measures heartbeat sounds by the unit of amplitude V over time t for a set period T. The heartbeat sounds measured through the measurement means 130 are transferred to the operation main body unit 120.

In the operation main body unit 120, the heartbeat sounds are converted into a digital signal through the A/D converter 124, and the digital signal is amplified through the amplifier 126. The converted and amplified heartbeat signal is operated through a stabilization determination step of the control unit 123 to determine whether the heartbeat is stabilized.

At the stabilization determination step, a distance between the points having an amplitude larger than a threshold value in the heartbeat signal is calculated, and whether the heartbeat is stabilized is determined according a result of comparing the calculated result and a set value stored in the storage unit 125.

For example, heartbeat sounds are measured by the measurement means 130 for ten second and converted into a computable signal by the operation main body unit 120. Then, the control unit 123 analyzes the time intervals between the points having a maximum amplitude (about 0.15V or higher) from the heartbeat signal and calculates an average value and a standard deviation of the time intervals. The average value and the standard deviation calculated by the control unit 123 are compared with an average value and a standard deviation stored in the storage unit 125. If the calculated standard deviation is within the range of the standard deviation stored in the storage unit 125, the heartbeat sounds are determined as stabilized heartbeat sounds, and if the calculated standard deviation is out of the range of the standard deviation stored in the storage unit 125, the heartbeat sounds are determined as heartbeat sounds that are not stabilized.

If the heartbeat sounds are determined as stabilized heartbeat sounds through the stabilization determination step of the control unit 123, a storing indication signal is displayed by the display unit 129, and the heartbeat sounds are stored in the storage unit 125 as a heartbeat signal to be played back. In addition, if the heartbeat sounds are determined as heartbeat sounds that are not stabilized through the stabilization determination step of the control unit 123, a re-measuring indication signal is displayed by the display unit 129, and a new heartbeat signal is measured again and operated. When the heartbeat signal is measured again, the measurement means 130 preferably measures the heartbeat signal by changing the point contacting with the body of the user.

The measurement means 130 measures two heartbeat sounds having a regular cycle, and the control unit 123 may determine whether the heartbeat is stabilized by comparing two heartbeat signals.

Another example of the stabilization determination step is operating two measured heartbeat signals and determining whether the heartbeat is stabilized according to a result of comparing the two operation values.

For example, two heartbeat sounds are measured in units of ten seconds by the measurement means 130 and converted into computable signals by the operation main body unit 120. Then, the control unit 123 analyzes the time intervals between the points having a maximum amplitude (about 0.15V or higher) from the two heartbeat signals and calculates an average value and a standard deviation of the time intervals. The calculated average values and standard deviations are compared with each other by the control unit 123, and if the difference is smaller than a predetermined value, the heartbeat sounds are determined as stabilized heartbeat sounds, and if the difference exceeds the predetermined value, the heartbeat sounds are determined as heartbeat sounds that are not stabilized.

Accordingly, a sense of psychological stability may be given to the infant more effectively by determining and storing heartbeat sounds of a mother that a baby may feel stably among the heartbeat sounds of the user.

The playback mode works when the user switches the mode change switch 141 of the switch unit 140 to the playback mode. In the playback mode, the stabilized heartbeat signal stored in the storage unit 125 in the measurement mode is repeatedly played back through the output unit 127. The output unit 127 outputs a sound amplified eight to twelve times as much as the sound measured by the measurement unit 130. A playback indication signal is displayed on the display unit 129. Like this, since heartbeat sounds of a mother is provided when a baby sleeps, sleep is effectively induced and the sleep state duration is extended owing to the sense of psychological stability that the mother is staying together with the baby.

When the heartbeat sounds are played back in the playback mode, heartbeat sounds synthesized with other sounds may be outputted. The sounds synthesized with the heartbeat sounds may be a wave sound, a vehicle engine sound or the like. The wave sound or the vehicle engine sound may be helpful for the baby to get a sense of psychological stability. The wave sound or the vehicle engine sound is measured in the measurement mode of the system for inducing sleep of an infant and recorded without an operation process by omitting the stabilization determination step of the measurement mode. When the heartbeat sounds synthesized with other sound are outputted through the output unit 127, the heartbeat sounds are outputted after being amplified eight to twelve times as much as the measured sound, and the synthesized wave sound or vehicle engine sound is not amplified and outputted as is the measured sound.

An experiment applying the system for inducing sleep of an infant according to the present invention has been performed on a seven-month old baby, who falls asleep in thirty five minutes in average in the mother's arms in normal times and sleeps for twenty minutes in average while being laid down on the floor, and it is confirmed that the time taken to fall asleep is thirty five minutes when the system for inducing sleep of an infant is applied, which is the same as the time taken to fall asleep when the baby stays together with the mother, and the time of maintaining the sleep is sixty minutes when the system for inducing sleep of an infant is applied, which is longer than the time of maintaining the sleep while the baby is laid down on the floor.

In addition, an experiment applying the system for inducing sleep of an infant according to the present invention has been performed on a three-month old baby, who falls asleep in twenty minutes in average in the mother's arms in normal times and sleeps for twenty minutes in average in normal times while being laid down on the floor, and it is confirmed that the time taken to fall asleep is twenty minutes when the system for inducing sleep of an infant is applied, which is the same as the time taken to fall asleep when the baby stays together with the mother, and the time of maintaining the sleep is thirty minutes when the system for inducing sleep of an infant is applied, which is longer than the time of maintaining the sleep while the baby is laid down on the floor.

Although the system for inducing sleep of an infant according to an embodiment of the present invention has been described with reference to the embodiments shown in the figures, it is merely illustrative, and those skilled in the art will be understood that various modifications and equivalent other embodiments can be made therefrom. Therefore, the true scope of the present invention should be defined by the technical spirit of the appended claims.

### INDUSTRIAL APPLICABILITY

Sleep is effectively induced and sleep state duration may be extended by giving a baby a sense of psychological stability that the mother is staying together with the baby by means of repeatedly providing the baby with heartbeat sounds of the mother stabilized by the system for inducing sleep of an infant according to the present invention as described above.

1. A system for inducing sleep of an infant, the system comprising:
a cushion main body, an operation main body unit provided inside the cushion main body, a measurement means connected to the operation main body unit to measure heartbeat of a human body, and a switch unit, wherein
the measurement means includes a hollow case opened toward one side and a measurement unit provided in an opening of the case to measure the heartbeat;
the operation main body unit includes a main body unit, a control unit provided in the main body unit, a storage unit connected to the control unit and provided in the main body unit, an output unit connected to the control unit and provided in the main body unit, and a display unit connected to the control unit and provided in the main body unit;
the switch unit is provided to be connected to the control unit; and
a heartbeat signal of the human body measured by the measurement means is stored in the storage unit, and outputted and repeatedly played back through the output unit by a playback command of a user received through the switch unit.

2. The system according to claim 1, wherein the switch unit includes a power switch, a mode change switch and a volume control switch, wherein if a measurement mode is selected by operating the mode change switch, whether the heartbeat is stabilized is determined by measuring the heartbeat signal measured by the measurement means and operating the measured heartbeat signal, and a result of the determination is displayed through the display unit.

3. The system according to claim 2, wherein if the user switches the mode change switch to a playback mode, the stabilized heartbeat signal measured and stored in the storage unit is repeatedly played back through the output unit.

4. The system according to claim 1, wherein the heartbeat signal is stored in the storage unit by way of a stabilization determination step, and at the stabilization determination step, the heartbeat signal measured by the measurement means is operated, and a result of the operation is compared with a set value stored in the storage unit, and if the result of the operation is within a range of the set value stored in the storage unit, a storing indication signal is displayed by the display unit, and the heartbeat signal is stored in the storage unit as a heartbeat signal to be played back, and if the result of the operation is out of the range of the set value, a re-measuring indication signal is displayed by the display unit, and a new heartbeat signal is measured again and operated.

5. The system according to claim 4, wherein at the stabilization determination step, the heartbeat signal is measured during two set periods and stored in the storage unit, an average value and a standard deviation of the two heartbeat signals are calculated, and whether the heartbeat is stabilized is determined by comparing the average values and the standard deviations of the two heartbeat signals.
